(19) United States
(12) Patent Application Publication
(10) Pub. No.: US 2024/0100117 A1
(43) Pub. Date: Mar. 28, 2024

(54) NUTRITION-BASED METHOD FOR MANAGING ANIMAL BEHAVIOR

(71) Applicant: Morgenstern & Associates, LLC, Morrow, OH (US)

(72) Inventor: Jeffrey Morgenstern, Morrow, OH (US)

(21) Appl. No.: 18/528,030

(22) Filed: Dec. 4, 2023

Related U.S. Application Data
(63) Continuation of application No. 17/568,390, filed on Jan. 4, 2022.

(51) Int. Cl. A61K 36/53; A23K 10/30; A23K 20/105; A23K 20/142; A23K 20/158; A23K 20/174; A23K 50/20; A61K 31/221; A61K 31/405; A61K 31/675

(52) U.S. Cl. CPC A61K 36/53; A23K 10/30; A23K 20/105; A23K 20/142; A23K 20/158; A23K 20/174; A23K 50/20; A61K 31/221; A61K 31/405; A61K 31/675

(57) ABSTRACT

A nutrition-based method for managing the behavior of a large, domesticated animal, comprising administering a nutritional formulation to a large, domesticated animal, wherein the nutritional formulation includes an effective amount of at least one suppressive neurotransmitter and an effective amount of at least one herbal estrous cycle effect moderator, and wherein the nutritional formulation moderates anxiety, reduces stress, and reduces erratic behavior in the large, domesticated animal based at least in part on the activity of the at least one suppressive neurotransmitter and the at least one herbal estrous cycle effect moderator.

### CROSS-REFERENCE TO RELATED APPLICATIONS

This patent application is a continuation of U.S. patent application Ser. No. 17/568,390 filed on Jan. 4, 2022 and entitled “Nutrition-Based Method for Managing Animal Behavior”, the disclosure of which is hereby incorporated by reference herein in its entirety and made part of the present U.S. utility patent application for all purposes.

### BACKGROUND

The disclosed inventive subject matter relates in general to nutritional products and formulations for use with animals and more specifically to a nutritional formulation for use with large, domesticated animals such as horses, wherein the nutritional formulation is administered as part of a system or method for managing the behavior the animal through moderation and mitigation of anxiety, reduction of stress, and reduction of erratic behavior and other undesirable animal behavior.

Anxiety, stress, and erratic behavior are significant and common problems in horses and other large domesticated animals. These problems can result in serious health issues, have negative effects on appetite, cause injuries to the animal, and cause injuries to caretakers or riders of the animal. Horses may be trained to perform in horse show events where animals perform various skills. Similarly, cattle may be trained for presentation at fairs and exposition competitions. Anxiety, stress, and erratic behavior in these situations may prevent the animals from demonstrating their true capabilities and may negatively affect the perception and value of the animals. Various supplements and treatments have been used to alleviate these problems, but none have proved particularly successful. In attempting to mitigate stress, certain supplements and treatments actually inhibit the animal's focus, further complicating problems. Therefore, improved nutrition-based methods and nutritional supplements are needed for effectively managing anxiety, stress, and erratic behavior in large domesticated animals without compromising focus and performance and without negatively impacting the health of the animal.

### SUMMARY

The following provides a summary of certain example implementations of the disclosed inventive subject matter. This summary is not an extensive overview and is not intended to identify key or critical aspects or elements of the disclosed inventive subject matter or to delineate its scope. However, it is to be understood that the use of indefinite articles in the language used to describe and claim the disclosed inventive subject matter is not intended in any way to limit the described inventive subject matter. Rather the use of “a” or “an” should be interpreted to mean “at least one” or “one or more”.

One implementation of the disclosed technology provides a first nutrition-based method for managing the behavior of a large, domesticated animal, comprising administering a nutritional formulation to a large, domesticated animal, wherein the nutritional formulation includes an effective amount of at least one suppressive neurotransmitter and an effective amount of at least one herbal estrous cycle effect moderator, and wherein the nutritional formulation moderates anxiety, reduces stress, and reduces erratic behavior in the large, domesticated animal based at least in part on the activity of the at least one suppressive neurotransmitter and the at least one herbal estrous cycle effect moderator.

The at least one suppressive neurotransmitter may include serotonin; L-tryptophan; 5-hydroxytryptophan; γ-aminobutyric acid; melatonin; or combinations thereof. The at least one suppressive neurotransmitter may comprise from about 0.1 to about 10.0% by weight of the formulation. The at least one herbal estrous cycle effect moderator may include chasteberry extract. The at least one suppressive neurotransmitter and the at least one herbal estrous cycle effect moderator may be dissolved in a liquid carrier. The liquid carrier may include a water-based component and an oil-based component. The nutritional formulation may further comprise at least one surfactant solubilizer, and the at least one surfactant solubilizer may include 12-hydroxy-9-cis-octadecenoic acid residues (ricinoleic acid residues); 12-hydroxy-9-cis-octadecenoic acid (ricinoleic acid); ethoxylated castor oil; or combinations thereof. The at least one surfactant solubilizer may comprise from about 5% to about 40% by weight of the formulation. The nutritional formulation may further comprise at least one emulsifier, and the at least one emulsifier may include lecithin; gum arabic; octenyl succinate starches; or combinations thereof. The nutritional formulation may further comprise at least one calming agent, and the at least one calming agent may include magnesium bisglycinate chelate; pyridoxine (B6); pyridoxine HCL; or combinations thereof. The nutritional formulation further may comprise at least one preservative, and the at least one preservative may include potassium sorbate, citric acid, or a combination thereof.

Another implementation of the disclosed technology provides a second nutrition-based method for managing the behavior of a large, domesticated animal, comprising administering a nutritional formulation to a large, domesticated animal, wherein the nutritional formulation includes an effective amount of at least one suppressive neurotransmitter and an effective amount of at least one herbal estrous cycle effect moderator, wherein the at least one suppressive neurotransmitter and the at least one herbal estrous cycle effect moderator have been dissolved in a liquid carrier, wherein the liquid carrier has a viscosity of less than about 2500 centipoises (cP), and wherein the liquid carrier includes a water-based component and an oil-based component, and wherein the nutritional formulation moderates anxiety, reduces stress, and reduces erratic behavior in the large, domesticated animal based at least in part on the activity of the at least one suppressive neurotransmitter and the at least one herbal estrous cycle effect moderator.

The at least one suppressive neurotransmitter may include serotonin; L-tryptophan; 5-hydroxytryptophan; γ-aminobutyric acid; melatonin; or combinations thereof. The at least one suppressive neurotransmitter may comprise from about 0.1 to about 10.0% by weight of the formulation. The at least one herbal estrous cycle effect moderator may include chasteberry extract. The nutritional formulation may further comprise at least one surfactant solubilizer, and the at least one surfactant solubilizer may include 12-hydroxy-9-cis-octadecenoic acid residues (ricinoleic acid residues); 12-hydroxy-9-cis-octadecenoic acid (ricinoleic acid); ethoxylated castor oil; or combinations thereof. The at least one surfactant solubilizer may comprise from about 5% to about 40% by weight of the formulation. The nutritional formulation may further comprise at least one emulsifier, and the at least one emulsifier may include lecithin; gum arabic; octenyl succinate starches; or combinations thereof. The nutritional formulation may further comprise at least one calming agent, and the at least one calming agent may include magnesium bisglycinate chelate; pyridoxine (B6); pyridoxine HCL; or combinations thereof. The nutritional formulation may further comprise at least one preservative, and the at least one preservative may include potassium sorbate, citric acid, or a combination thereof.

Still another implementation of the disclosed technology provides a third a nutrition-based method for managing the behavior of a large, domesticated animal, comprising administering a nutritional formulation to a large, domesticated animal, wherein the nutritional formulation includes an effective amount of at least one suppressive neurotransmitter selected from the group consisting of serotonin; L-tryptophan; 5-hydroxytryptophan; γ-aminobutyric acid; and melatonin; an effective amount of at least one herbal estrous cycle effect moderator; and an effective amount of at least one calming agent, wherein the at least one calming agent is selected from the group consisting of magnesium bisglycinate chelate; pyridoxine (B6); and pyridoxine HCL, wherein the at least one suppressive neurotransmitter and the at least one herbal estrous cycle effect moderator have been dissolved in a liquid carrier, wherein the liquid carrier has a viscosity of less than about 2500 centipoises (cP), and wherein the liquid carrier includes a water-based component and an oil-based component, and wherein the nutritional formulation moderates anxiety, reduces stress, and reduces erratic behavior in the large, domesticated animal based at least in part on the activity of the at least one suppressive neurotransmitter, the at least one herbal estrous cycle effect moderator, and the at least one calming agent.

The at least one herbal estrous cycle effect moderator may include chasteberry extract. The nutritional formulation may further comprise at least one surfactant solubilizer, wherein the at least one surfactant solubilizer includes 12-hydroxy-9-cis-octadecenoic acid residues (ricinoleic acid residues); 12-hydroxy-9-cis-octadecenoic acid (ricinoleic acid); ethoxylated castor oil; or combinations thereof, and wherein the at least one surfactant solubilizer comprises from about 5% to about 40% by weight of the formulation; at least one emulsifier, wherein the at least one emulsifier includes lecithin; gum arabic; octenyl succinate starches; or combinations thereof; and at least one preservative, wherein the at least one preservative includes potassium sorbate, citric acid, or a combination thereof.

It should be appreciated that all combinations of the foregoing concepts and additional concepts discussed in greater detail below (provided such concepts are not mutually inconsistent) are contemplated as being part of the inventive subject matter disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the drawings and associated descriptions are to be regarded as illustrative and not restrictive in nature.

### DETAILED DESCRIPTION

Example implementations are described below. Although the following detailed description contains many specifics for the purposes of illustration, a person of ordinary skill in the art will appreciate that many variations and alterations to the following details are within the scope of the disclosed inventive subject matter. Accordingly, the following example implementations are set forth without any loss of generality to, and without imposing limitations upon, the claimed subject matter.

Implementations of the disclosed methods and formulations provide improved calming supplements for use with animals such as horses and livestock. These nutritional supplements may be used daily to maintain desired health and behavior without negatively impacting the overall physiological health of the animal. The supplements may also be beneficial for special occurrences in which the animal is outside of its normal environment and is exposed to unusual or unexpected stimuli. In such unusual or atypical environments, the supplements moderate the animal's anxiety and erratic behavior without compromising its focus or performance. Certain implementations of the disclosed inventive subject matter provide an nutritional supplement in a liquid delivery vehicle that may be more readily incorporated into an animal feed, is more controllable regarding dosing and ingestion, and may be provided in more environmentally favorable packaging. As described below, causes of anxiety, stress, and erratic behavior in horses and other large animals may include hormonal factors and environmental factors.

With regard to hormonal factors, the estrous cycle in mares is seasonal in most locations and is regulated primarily by the amount of daylight. As the days lengthen in the spring, light causes the mare's pineal gland to reduce serotonin secretion. Serotonin is a neurotransmitter derived from the amino acid tryptophan and is a precursor to melatonin. The production of melatonin is important because it has a significant effect on the estrous cycle, sleep-wake cycles, and other phenomena having a circadian rhythm. Increased daylight also causes the pineal gland to release gonadotropin-releasing hormone (GnRH) from the hypothalamus. GnRH in turn stimulates the pituitary gland to release follicle stimulating hormone (FSH) and luteinizing hormone (LH). As the season progresses, the release of FSH and LH evolves into a cycle. Hormones regulate the development of follicles in the ovary, ovulation, and the development of corpora lutea, which replaces the ovulated follicle. Developing follicles release estrogen, the hormone that instigates higher-stress behavior in a mare, when in heat. A mare's behavioral response to estrogen is related to mating and can include squealing, aggressive behavior toward other horses, tail swishing, and frequent urination. In general, the horse becomes less trainable in this state and less likely to perform well in a competitive event. Estrogen production ceases just prior to ovulation and once the development of corpora lutea begins, it releases progesterone, the hormone associated with calmer behavior.

With regard to environment factors, when at home, a horse is typically in a relatively quiet, stable environment where it is the only horse or one of a small, familiar group of horses. The horse typically is exposed to the same person or persons who feed and care for the horse daily. It is in this environment that the horse is trained to perform certain skills. The consistent familiarity of this environment provides a level of comfort to the horse. Occasionally, however, the horse's owner may wish to demonstrate the horse's skills in a horse show, which usually requires transporting the horse to a remote location in a horse trailer. Confinement in the horse trailer, sometimes for extended periods, may cause adrenaline, stress, and anxiety levels to increase significantly. Transport-related exposure to stopping, starting, turns, road bumps, traffic construction noise, etc. may further exacerbate anxiety and stress. Once reaching the destination of a horse show, the horse is then placed in a challenging environment that may include new barns, stalls, event buildings, many unfamiliar horses, many unfamiliar people, noise, etc. Such dramatic changes in the horse's environment may cause an increase in adrenaline, stress, anxiety, and erratic behavior, which may compromise the horse's ability to focus and perform in events at a level consistent with what it has demonstrated in its more natural home environment.

Animal supplements are commonly provided in solid form such as, for example, powder, granules, pellets, etc., which can be mixed with feed. This can be an effective delivery system for supplements that do not require accurate dosing, but does not necessarily provide sufficient control for the delivery of concentrated active ingredients. Additionally, many animals do not uniformly consume their feed and selectively or preferentially eat the ingredients, often leaving behind supplements that have been added.

Animal supplements are also commonly provided in single dose applicators that contain a predetermined volume of a gel or paste substance. The gel is applied to an animal's gum or tongue and is subsequently swallowed. While this approach is more controlled, there is still uncertainty regarding if and when a supplement has been swallowed because portions of the supplement may remain in the mouth for some period of time or may be discarded by the animal. Pre-measured single applicators also lack flexibility required for adjusting dosage to account for the size of horse, particularly high-stress events, and other unexpected or unpredictable events or factors. There are also environmental waste concerns associated with the use of plastic, single dose applicators.

Implementations of the disclosed formulations are provided as solids, semi-solids, and liquids containing nutritional ingredients and active ingredients that function as suppressive neurotransmitters and calming agents. The term liquid, as used herein, refers to the relatively low viscosity of the formulation, which allows the formulation to flow from packaging, such as a bottle, and be dispensed in any desired volume. The formulation may be given directly to the animal (e.g. by syringe) or by mixing the liquid with the animal's feed, in a manner not possible with exiting solid or gel-type supplements.

Viscosity is the measure of a fluid's resistance to gradual deformation by shear stress or tensile stress. For liquids, viscosity corresponds to the informal concept of “thickness”. For example, syrup has a higher viscosity than water. For general reference, glycerin at room temperature has a viscosity of about 950 centipoises (cP); castor oil has a viscosity of about 650 cP; linseed oil has a viscosity of about 35 cP; and water has a viscosity of about 1 cP. Various example implementations of the disclosed liquid nutritional supplement have a viscosity less than about 2500 cP; less than about 1000 cP; less than about 500 cP; less than about 100 cP; or less than about 50 cP.

Example implementations of the disclosed formulation include at least one compound that is or has the effect of a suppressive neurotransmitter. An effective suppressive neurotransmitter may increase the amount of serotonin in the blood stream of an animal and may mitigate the effects of the estrous cycle, particularly in mares, thereby calming the animal. In certain non-limiting examples, the suppressive neurotransmitter is serotonin; L-tryptophan; 5-hydroxytryptophan; γ-aminobutyric acid; melatonin, and combinations thereof. Serotonin is a neurotransmitter associated with a feeling of calm. Serotonin is also a precursor to melatonin, which has a significant effect on the estrous cycle, sleep-wake cycles, and other physiological phenomena associated with circadian rhythms. The amino acids 5-hydroxytryptophan (5-HTP) and L-tryptophan are serotonin precursors. 5-HTP offers some advantages over L-tryptophan because it crosses the blood-brain barrier at a higher rate, is converted into serotonin more efficiently than L-tryptophan and may exhibit a more pronounced antidepressant effect. Example implementations of the disclosed formulation include at least 100 mg of L-tryptophan or at least 100 mg of hydroxytryptophan (5-HTP). In implementations that include a suppressive neurotransmitter, the suppressive neurotransmitter represents about 0.1 percent to about 10 percent by weight of the formulation; about 0.5 percent to about 5.0 percent of the formulation; or about 1.0 percent to about 3.5 percent by weight of the formulation.

Certain versions of the disclosed formula may comprise at least one surfactant solubilizer. The disclosed formula may be delivered in a unique liquid formulation which overcomes the limitations of prior art solid and gel supplement forms. The liquid formulation is achieved through the use of a solubilizer that includes one or more surfactants that are substantially or completely water soluble but that also exhibits solubility in oil. These surfactants are used to solubilize an oil in water or another hydrophilic medium. The solubilized material is of such a small particle size that the solution appears either completely transparent or lightly translucent.

In some implementations, and by way of non-limiting example, the surfactant solubilizer may include 12-hydroxy-9-cis-octadecenoic acid residues (ricinoleic acid residues); 12-hydroxy-9-cis-octadecenoic acid (ricinoleic acid); ethoxylated castor oil; or combinations thereof. In one example implementation, ethoxylated castor oil is prepared by reacting 35 moles of ethylene oxide with one mole of castor oil which reacts as a non-ionic detergent in solution. This approach results in compatibility with other ingredients and can be used with fat-soluble vitamins and essential oils. In an example implementation, an ethoxylated castor oil is used as the solubilizer and is mixed with calming agents, herbal ingredients, vitamin and general health ingredients, flavors, and preservatives to produce an effective liquid supplement suitable for calming the animal. In one example implementation, Kolliphor® EL (formerly Cremphor® EL), manufactured by BASF, is the surfactant solubilizer. In example implementations that include the surfactant solubilizer, the solubilizer represents from about 5 percent to about 40 percent by weight of the formulation; from about 10 percent to about 30 percent of the formulation; or from about 15 percent to about 25 percent by weight of the formulation.

In other implementations, a liquid formulation is achieved through the use of an emulsifier rather than a solubilizer. The use of an emulsifier is likely to be more effective if other ingredients in the formulation comprise larger, lipophilic molecules or if the oil phase concentration is relatively high. Solubilizers typically work well up to 2% oil phase concentration while emulsifiers are typically effective up to 50% oil phase concentration. Examples of desirable ingredients include oil-soluble vitamins, such as A, D, E and K, which are associated with health and well-being. These vitamins tend to exhibit low water-solubility, chemical instability, and low and variable oral bioavailability. Accordingly, these vitamins cannot easily be incorporated into aqueous solutions. Emulsions may be used to overcome these challenges and provide a liquid formulation. Example emulsifiers include lecithin; gum arabic; octenyl succinate starches; and combinations thereof.

Implementations of the disclosed formulation that are intended specifically for use with mares may include an herbal estrous cycle effect moderating agent. As previously described, the estrous cycle of a mare creates significant fluctuations in hormones, with corresponding effects on behavior. To mitigate such issues, the estrous cycle of a mare can be suppressed through the administration of progesterone, injections of oxytocin, or the insertion of a glass ball into the mare's uterus. However, many horse owners prefer to avoid the cost and potential negative side effects associated with administering progesterone or using injections for estrous cycle suppression. Herbal ingredients or supplements are an appealing alternative because such compositions may moderate the effects of the estrous cycle in a more natural manner by potentiating progesterone. Such compositions may work in an additive manner, where prolonged daily use may cause an increasing, moderating effect as the body of the horse absorbs more of the supplement or supplements.

As will be appreciated by one of ordinary skill, the following herbs and herb combinations are known to moderate estrous cycle effects: (i) chasteberry extract; (ii) oregano leaf extract; (iii) vervain, cramp bark, yarrow, rosemary, and lemon balm; (iv) raspberry leaf, and blue cohosh; (v) raspberry leaf and blue cohosh; (vi) dandelion leaves; (vii) chamomile and valerian; and (viii) dong quai. In the scientific literature, chasteberry extract has demonstrated to regulate the pituitary gland which regulates hormones in horses. Chasteberry essentially acts as a progesterone precursor, thus moderating hormone fluctuations related to the estrous cycle. Oregano leaf extract is a known emmenagogue which can balance the estrous cycle and that has anti-inflammatory and pain-relieving properties. Vervain, cramp bark, yarrow, rosemary, and lemon balm may relax uterine muscle and exhibit antispasmodic properties. Raspberry leaf and blue cohosh are alkaloids that can strengthen the uterus and mitigate cramping. Dandelion leaves are a diuretic that can relieve water retention associated with the estrous cycle and that may help the liver function properly, thereby improving overall hormone balance. Chamomile and valerian are herbs that relieve anxiety and stress. Chamomile exhibits anti-inflammatory properties and may mitigate cramps and bloating. Dong quai contains ferulic acid, which may relax uterine muscle. In example implementations that include an estrous cycle moderating agent, the agent represents from about 1 percent to about 30 percent by weight of the formulation; from about 3 percent to about 20 percent by weight of the formulation; or from about 5 percent to about 15 percent by weight of the formulation.

Certain implementations of the disclosed formulation include at least one animal calming component or agent. By way of non-limiting example, the animal calming agent may be magnesium bisglycinate chelate; pyridoxine (vitamin B6); pyridoxine HCL; or combinations thereof. Magnesium regulates many body processes, including muscle and nerve function, blood sugar levels, and blood pressure and supports the making of protein, bone, and DNA. Adequate levels of magnesium are also necessary for maintaining proper levels of serotonin. Magnesium bisglycinate chelate provides magnesium in a form that is easily absorbed and that is easily digestible. A chelate includes a metal ion and a chelating agent that creates multiple soluble bonds with the ion. This chemical structure facilitates the dissolution of chelates; therefore, the use of a chelate in a supplement may significantly improve the absorption and utilization of other ingredients such as magnesium. Combinations of pyridoxine and pyridoxine HCL provide water-soluble blends of vitamin B6, which cannot be produced in the body and must be obtained from food or supplements. Vitamin B6 is important in protein, fat, and carbohydrate metabolism and in the creation of red blood cells and neurotransmitters. Vitamin B6 is also involved in mood regulation in part because the vitamin is necessary for creating neurotransmitters that regulate emotions, including serotonin, dopamine, and gamma-aminobutyric acid (GABA). A 1995 “Journal of Neural Transmission” study demonstrated that supplementation with vitamin B6 increased the conversion of 5-HTP to serotonin in primate animal models. Therefore, combining this compound with a serotonin precursor such as hydroxytryptophan (5-HTP) may be particularly effective in increasing serotonin and providing a reliable calming supplement for animals. In example implementations that include the animal calming agent, the agent represents about 0.1 percent to about 10 percent by weight of the formation; about 0.5 percent to about 5 percent by weight of the product or about 1 percent to about 3 percent.

Implementations of the disclosed formulation may include other components, ingredients, or compounds found in solid, paste, gel or liquid nutritional supplements such as various flavors and preservatives. Non-limiting examples of such flavors and preservatives include glycerin, soybean oil, sodium copper chlorophyllin, natural and artificial flavoring, potassium sorbate, and citric acid.

### NON-LIMITING WORKING EXAMPLES

The following examples are provided to illustrate particular features of certain examples, but the scope of the claims should not be limited to those features exemplified. Ingredients listed in the following examples are commercially available from a variety of vendors and sources such as, for example, BASF and DuPont, other commercial vendors of chemical ingredients and nutritional ingredients used in food supplements and nutraceuticals.

### Example 1

| Item | | Amount (% |
|---|---|---|
| (for blending) | INGREDIENT | by weight) |
| 1 | Water RO/Deionized | 39.19% |
| 1 | Vanilla Flavor—Artificial | 0.52% |
| 2 | Chasteberry Extract | 10.16% |
| 2 | Potassium Sorbate | 0.3% |
| 2 | Magnesium Biglycinate | 0.41% |
| 2 | Citric Acid | 0.22% |
| 2 | L-5 Hydroxytryptophan | 1.43% |
| | (Griffonia Powder) | |
| 2 | Pyridoxine HCL (B6) | 1.32% |
| 2 | Stevia Extract | 0.52% |
| 2 | Sodium Copper Chlorophyllin | 0.000001% |
| 2 | Crushed Berry Flavor—Natural | 0.53% |
| | and Artificial | |
| 3 | Soybean Oil Expellers | 10.72% |
| 3 | Glycerin | 15.08% |
| 3 | Kolliphor Castor Oil | 19.05% |
| 4 | Placental Powder (Porcine) | 0.1% |
| 4 | Adrenal Cortex Powder (Porcine) | 0.1% |
| 4 | Ovarian Substance (Porcine) | 0.1% |
| 4 | Oregano Leaf Extract | 0.24% |
| Total | | 100% |
| Serving Size | | 32.1g |

Example 1 (above) may be made by the following example method, which results in a liquid nutritional supplement for use with animals such as horses: (i) heat items (1) to between 100-140 degrees Fahrenheit; (ii) mix dry items (2) in a separate vessel for 5-7 minutes and add to items (1); (iii) mix for 50-70 minutes; (iv) in a separate heated mixer, add items (3) and heat to 100-140 degrees Fahrenheit; (v) mix dry items (4) in a separate bowl for 5 to 7 minutes and add to items (3); (vi) xix for 6-10 minutes; (vii) add blend #1 (water based ingredients) to blend #2 (oil based ingredients); and (viii) mix for an additional 6-10 minutes. The examples below are made in a similar manner under similar conditions.

### Example 2

| | Amount |
|---|---|
| INGREDIENT | (% by wt) |
| Kolliphor EL Polyoxyl 35 Castor Oil NF EP | 14.0% |
| Glycerin | 18.0% |
| Soybean Oil Expellers | 12.7% |
| Vervain extract | 5.3% |
| L-Hydroxytryptophan | 5.0% |
| Pyridoxine HCL—Vitamin B6 Blend (82.3%) | 1.3% |
| Stevia Extract | 0.5% |
| Crushed Berry | 0.5% |
| Vanilla Flavor Artificial | 0.5% |
| Magnesium Biglycinate Chelate, Buffered | 0.4% |
| Potassium Sorbate | 0.3% |
| Oregano Leaf Extract (2%) | 5.2% |
| Citric Acid | 0.2% |
| DI Water | -q.s- |
| Viscosity | <50 cP |

### Example 3

| | Amount |
|---|---|
| INGREDIENT | (% by wt) |
| Kolliphor EL Polyoxyl 35 Castor Oil NF EP | 19.0% |
| Glycerin | 15.0% |
| Soybean Oil Expellers | 10.7% |
| Chasteberry Extract | 10.2% |
| 5 hydroxytryptophan (98%) | 3.0% |
| Pyridoxine HCL—Vitamin B6 Blend (82.3%) | 1.3% |
| Stevia Extract | 0.5% |
| Crushed Berry | 0.5% |
| Vanilla Flavor Artificial | 0.5% |
| Magnesium Biglycinate Chelate Buffered | 0.4% |
| Potassium Sorbate | 0.3% |
| Oregano Leaf Extract (2%) | 0.2% |
| Citric Acid | 0.2% |
| DI Water | -q.s- |
| Viscosity | <50 cP |

### Example 4

| | Amount |
|---|---|
| INGREDIENT | (% by wt) |
| Kolliphor EL Polyoxyl 35 Castor Oil NF EP | 25.0% |
| Glycerin | 25.0% |
| Chamomile Oil | 5.2% |
| Serotonin | 1.5% |
| Pyridoxine HCL (82.3%) | 1.3% |
| Stevia Extract | 0.5% |
| Crushed Berry | 0.5% |
| Vanilla Flavor Artificial | 0.5% |
| Magnesium Biglycinate Chelate Buffered | 0.4% |
| Potassium Sorbate | 0.3% |
| Oregano Leaf Extract (2%) | 5.2% |
| Citric Acid | 0.2% |
| DI Water | -q.s- |
| Viscosity | <50 cP |

All literature and similar material cited in this application, including, but not limited to, patents, patent applications, articles, books, treatises, and web pages, regardless of the format of such literature and similar materials, are expressly incorporated by reference in their entirety. Should one or more of the incorporated references and similar materials differs from or contradicts this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about”, if or when used throughout this specification describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed inventive subject matter. Various functions and elements described herein may be partitioned differently from those shown without departing from the scope of the disclosed inventive subject matter. Generic principles defined herein may be applied to other implementations. Different numbers of a given module or unit may be employed, a different type or types of a given module or unit may be employed, a given module or unit may be added, or a given module or unit may be omitted.

Regarding this disclosure, the term “a plurality of” refers to two or more than two. Unless otherwise clearly defined, orientation or positional relations indicated by terms such as “upper” and “lower” are based on the orientation or positional relations as shown in the figures, only for facilitating description of the present invention and simplifying the description, rather than indicating or implying that the referred devices or elements must be in a particular orientation or constructed or operated in the particular orientation, and therefore they should not be construed as limiting the present invention. The terms “connected”, “mounted”, “fixed”, etc. should be understood in a broad sense. For example, “connected” may be a fixed connection, a detachable connection, or an integral connection; a direct connection, or an indirect connection through an intermediate medium. For an ordinary skilled in the art, the specific meaning of the above terms in the present invention may be understood according to specific circumstances.

It should be appreciated that all combinations of the foregoing concepts and additional concepts discussed in greater detail herein (provided such concepts are not mutually inconsistent) are contemplated as being part of the disclosed inventive subject matter. In particular, all combinations of claimed subject matter appearing at the end of this disclosure are contemplated as being part of the inventive subject matter disclosed herein. While the disclosed inventive subject matter has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed inventive subject matter in its broader aspects is not limited to any of the specific details, representative devices and methods, and/or illustrative examples shown and described. Accordingly, departures may be made from such details without departing from the spirit or scope of the general inventive concept.

What is claimed:

1. A nutrition-based method for managing the behavior of a large, domesticated animal, comprising
(a) administering a nutritional formulation to a large, domesticated animal,
(b) wherein the nutritional formulation includes:
i) an effective amount of a suppressive neurotransmitter; and
ii) an effective amount of an herbal estrous cycle effect moderator, and
(c) wherein the nutritional formulation moderates anxiety, reduces stress, and reduces erratic behavior in the large, domesticated animal based on the combined activity of the suppressive neurotransmitter and the herbal estrous cycle effect moderator.

2. The method of claim 1, wherein the suppressive neurotransmitter is selected from the group consisting of serotonin; L-tryptophan; 5-hydroxytryptophan; γ-aminobutyric acid; and melatonin.

3. The method of claim 1, wherein the suppressive neurotransmitter comprises from about

0. 1 to about 10.0% by weight of the formulation.

4. The method of claim 1, wherein the herbal estrous cycle effect moderator includes chasteberry extract.

5. The method of claim 1, wherein the suppressive neurotransmitter and the herbal estrous cycle effect moderator are dissolved in a liquid carrier.

6. The method of claim 5, wherein the liquid carrier includes a water-based component and an oil-based component.

7. The method of claim 1, wherein the nutritional formulation further comprises a surfactant solubilizer selected from the group consisting of 12-hydroxy-9-cis-octadecenoic acid residues (ricinoleic acid residues); 12-hydroxy-9-cis-octadecenoic acid (ricinoleic acid); and ethoxylated castor oil, and wherein the surfactant solubilizer comprises from about 5% to about 40% by weight of the formulation.

8. The method of claim 1, wherein the nutritional formulation further comprises an emulsifier selected from the group consisting of lecithin; gum arabic; and octenyl succinate starches.

9. The method of claim 1, wherein the nutritional formulation further comprises a calming agent selected from the group consisting of magnesium bisglycinate chelate; pyridoxine (B6); and pyridoxine HCL.

10. The method of claim 1, wherein the nutritional formulation further comprises a preservative that includes potassium sorbate or citric acid.

11. A nutrition-based method for managing the behavior of a large, domesticated animal, comprising:
(a) administering a nutritional formulation to a large, domesticated animal,
(b) wherein the nutritional formulation includes:
i) an effective amount of a suppressive neurotransmitter, and
ii) an effective amount of an herbal estrous cycle effect moderator,
(c) wherein the suppressive neurotransmitter and the herbal estrous cycle effect moderator are dissolved in a liquid carrier that has a viscosity of less than about 2500 centipoises (cP) and that has a water-based component and an oil-based component, and
(d) wherein the nutritional formulation moderates anxiety, reduces stress, and reduces erratic behavior in the large, domesticated animal based on the combined activity of the suppressive neurotransmitter and the herbal estrous cycle effect moderator.

12. The method of claim 11, wherein the suppressive neurotransmitter is serotonin; L-tryptophan; 5-hydroxytryptophan; γ-aminobutyric acid; or melatonin; and wherein the suppressive neurotransmitter comprises from about 0.1 to about 10.0% by weight of the formulation.

13. The method of claim 11, wherein the herbal estrous cycle effect moderator includes chasteberry extract.

14. The method of claim 11, wherein the nutritional formulation further comprises a surfactant solubilizer that includes 12-hydroxy-9-cis-octadecenoic acid residues (ricinoleic acid residues); 12-hydroxy-9-cis- octadecenoic acid (ricinoleic acid); orethoxylated castor oil; and that comprises from about 5% to about 40% by weight of the formulation.

15. The method of claim 11, wherein the nutritional formulation further comprises anemulsifier selected from the group consisting of lecithin; gum arabic; and octenyl succinate starches.

16. The method of claim 11, wherein the nutritional formulation further comprises a calming agent that includes magnesium bisglycinate chelate; pyridoxine (B6); or pyridoxine HCL.

17. The method of claim 11, wherein the nutritional formulation further comprises a preservative that includes potassium sorbate or citric acid.

18. A nutrition-based method for managing the behavior of a large, domesticated animal, comprising:
(a) administering a nutritional formulation to a large, domesticated animal,
(b) wherein the nutritional formulation includes:
i) an effective amount of a suppressive neurotransmitter selected from the group consisting of serotonin; L-tryptophan; 5-hydroxytryptophan; γ-aminobutyric acid; and melatonin;
ii) an effective amount of an herbal estrous cycle effect moderator, and
iii) an effective amount of a calming agent, wherein the calming agent is selected from the group consisting of magnesium bisglycinate chelate; pyridoxine (B6); and pyridoxine HCL,
(c) wherein the suppressive neurotransmitter and the herbal estrous cycle effect moderator are dissolved in a liquid carrier that has a viscosity of less than about 2500 centipoises (cP) and that includes a water-based component and an oil-based component, and
(d) wherein the nutritional formulation moderates anxiety, reduces stress, and reduces erratic behavior in the large, domesticated animal based on the combined activity of the suppressive neurotransmitter, the herbal estrous cycle effect moderator, and the calming agent.

19. The method of claim 18, wherein the herbal estrous cycle effect moderator includes chasteberry extract.

20. The method of claim 18, wherein the nutritional formulation further comprises:
(a) a surfactant solubilizer that includes 12-hydroxy-9-cis-octadecenoic acid residues (ricinoleic acid residues); 12-hydroxy-9-cis-octadecenoic acid (ricinoleic acid);
orethoxylated castor oil; wherein the surfactant solubilizer comprises from about 5% to about 40% by weight of the formulation;
(b) an emulsifier selected from the group consisting of lecithin; gum arabic; and octenyl succinate starches; and
(c) a preservative that includes potassium sorbate or citric acid.
